(19) United States
(12) Patent Application Publication
(10) Pub. No.: US 2009/0012625 A1
(43) Pub. Date: Jan. 8, 2009

(54) Porous biomaterial-filler composite and method for making the same

(76) Inventors: Jackie Y. Ying, Singapore (SG); Shona Pek, Singapore (SG); Shujun Gao, Singapore (SG); Mohamed Shariff Arshad, Singapore (SG); Pei Lin Mao, Singapore (SG)

Correspondence Address: Medlen & Carroll LLP, 101 Howard Street Suite 350, San Francisco, CA, 94105, US

(21) Appl. No.: 11/662,737

(22) PCT Filed: Sep. 14, 2004
(86) PCT No.: PCT/SG04/00294

§ 371 Date: Sep. 25, 2008

(51) Int. Cl. A61F 2/28

(52) U.S. Cl. 623/23.63; 623/23.51; 623/23.61

(57) ABSTRACT

A porous biomaterial-filler composite comprising a biomaterial, such as collagen, interspersed with a calcium phosphate-type filler material. The porosity of the composite is similar to that of natural bone and can feature a pore size ranging from a few nanometres to greater than 100 microns. Scaffolds prepared from the biomaterial-filler composite are suitable for resorbable bone substitute materials.

### TECHNICAL FIELD

The present invention relates to a porous biomaterial-filler composite, a method of preparation, and uses thereof. In one particular embodiment, the present invention relates to a collagen-inorganic scaffold.

### BACKGROUND OF THE INVENTION

30 to 35% of bone is composed of organic material (on a dry weight basis). Of this amount, about 95% is collagen. The remaining organic substances are chondroitin sulfate, keratin sulfate and phospholipids. 65 to 70% of bone is composed of inorganic substances. Almost all of these inorganic substances are composed of hydroxyapatite

When large amounts of lost bone need replacement, this is usually achieved by a variety of grafts or permanent alloy implants. Such grafting and implants are sometimes not desirable as they may not have sufficient strength to support an active lifestyle or sufficient bioactivity to promote cell attachment and proliferation. Many implants used are also not resorbable by natural tissue and cannot be tunable with respect to their mechanical properties or degradation rates.

Collagen has a low immunogenicity, is bioabsorbable and is a naturally occurring structural protein to which cells can attach, interact with and degrade. Collagen sponges and foams have been used as hemostatic agents, as scaffolds for tissue repair and as a support for cell growth. To date, however, no implant or collagen sponge has had the same or similar properties to that of natural bone.

### OBJECT OF THE INVENTION

It is an object of the present invention, at least in preferred embodiments, to overcome or substantially ameliorate at least one of the above disadvantages.

### SUMMARY OF THE INVENTION

According to a first aspect of the present invention, there is provided a porous biomaterial-filler composite wherein the composite comprises biomaterial interspersed with a filler and wherein the composite has a porosity close to that of natural bone.

According to a second aspect of the present invention, there is provided a porous biomaterial-filler composite wherein the composite comprises biomaterial interspersed with a filler and wherein the composite includes pores having a size greater than about 100 microns, pores having a size between about 30 and about 50 microns and nanometer sized pores.

According to a third aspect of the present invention, there is provided a porous s biomaterial-filler composite wherein the composite comprises biomaterial interspersed with a filler and wherein the dry weight ratio of biomaterial to filler is in the range 1:3 to 2:1.

In one embodiment, the composite comprises by dry weight 25 to 65 wt % biomaterial and 35 to 75 wt % filler.

According to a fourth aspect of the present invention, there is provided a porous biomaterial-filler composite wherein the composite comprises biomaterial interspersed with a filler wherein the amount of biomaterial is at least about 25 wt %.

In one embodiment of the third or fourth aspects, the composite may have a porosity close to that of bone.

According to a fifth aspect of the present invention, there is provided a porous biomaterial-filler composite wherein the composite comprises biomaterial interspersed with a filler and wherein the composite filler has been prepared using a sol-gel method.

According to a sixth aspect of the present invention, there is provided a method of preparing a porous biomaterial-filler composite comprising
combining biomaterial, a liquid and a filler to form a mixture,
homogenizing the mixture to form a slurry, and
freeze-drying the slurry to form a porous biomaterial-filler composite.

According to a seventh aspect of the present invention, there is provided a method of preparing a porous biomaterial-filler composite comprising
combining biomaterial and a liquid to form a mixture,
homogenizing the mixture to form a slurry,
adding a filler to the slurry;
further homogenizing the slurry; and
freeze-drying the slurry to form a porous biomaterial-filler composite.

According to an eighth aspect of the present invention, there is provided a method of preparing a porous biomaterial-filler composite comprising
combining a filler and a liquid to form a mixture,
homogenizing the mixture to form a slurry,
adding a biomaterial to the slurry;
further homogenizing the slurry; and
freeze-drying the slurry to form a porous biomaterial-filler composite

In one embodiment, the liquid is water or any other liquid capable of forming a slurry. In one embodiment when the biomaterial is collagen, the liquid is an acid.

According to a ninth aspect of the present invention, there is provided a method of preparing a porous biomaterial-filler composite comprising
combining biomaterial, a liquid and a filler to form a mixture,
homogenizing the mixture to form a slurry, and
freeze-drying the slurry to form a porous biomaterial-filler composite, wherein the liquid is an acid and the amount of biomaterial in the slurry is in the range of up to about 10 g biomaterial per 100 ml of up to a 1000 mM acid.

According to a tenth aspect of the present invention, there is provided a method of preparing a porous biomaterial-filler composite comprising
combining biomaterial and a liquid to form a mixture,
homogenizing the mixture to form a slurry,
adding a filler to the slurry;
further homogenizing the slurry; and
freeze-drying the slurry to form a porous biomaterial-filler composite, wherein the liquid is an acid and the amount of biomaterial in the slurry is in the range of up to about 10 g biomaterial per 100 ml of up to a 1000 mM acid.

According to an eleventh aspect of the present invention, there is provided a method of preparing a porous biomaterial-filler composite comprising
combining filler and a liquid to form a mixture,
homogenizing the mixture to form a slurry,
adding a biomaterial to the slurry;
further homogenizing the slurry; and
freeze-drying the slurry to form a porous biomaterial-filler composite, wherein the liquid is an acid and the amount of biomaterial in the slurry is in the range of up to about 10 g biomaterial per 100 ml of up to a 1000 mM acid.

According to a twelfth aspect, there is provided a composite prepared by the method of the sixth, seventh, eighth, ninth, tenth or eleventh aspects.

### DEFINITIONS

The following definitions are intended as general definitions and should in no way limit the scope of the present invention to those terms alone, but are put forth for a better understanding of the following description.

Unless the context requires otherwise or specifically stated to the contrary, integers, steps, or elements of the invention recited herein as singular integers, steps or elements clearly encompass both singular and plural forms of the recited integers, steps or elements.

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

All the references cited in this application are specifically incorporated by reference and are incorporated herein in their entirety.

In the context of this specification, the term “biomaterial” refers to any material which is suitable for introduction into a living organism such as a mammal including a human. The biomaterial is suitably non-toxic and bioabsorbable when introduced into a living organism and any degradation products of the biomaterial are also suitably non-toxic to the organism. The biomaterial may be derived from an organism, or may be a synthetic variant.

### BRIEF DESCRIPTION OF THE DRAWINGS

A preferred form of the present invention will now be described by way of example with reference to the accompanying drawings wherein:

FIG. 1 is a schematic diagram of a suitable method of preparing a composite material (bone scaffold) of the present invention;

FIG. 2 is a set of Scanning Electron Micrographs of collagen suitable for use in the present invention;

FIG. 3 is a set of Scanning Electron Micrographs of hydroxyapatite suitable for use in the present invention;

FIGS. 4A, 4B, 4C and 4D are graphs of stress with respect to strain of various porous scaffolds in accordance with the present invention;

FIG. 5 is a set of Scanning Electron Micrographs of trabecular bone at various resolutions;

FIG. 6 is a set of Scanning Electron Micrographs of a collagen-inorganic scaffold in accordance with one embodiment of the present invention at various resolutions;

FIG. 7 is a set of Scanning Electron Micrographs of a collagen-inorganic scaffold in accordance with another embodiment of the present invention at various resolutions;

FIG. 8 is a set of Scanning Electron Micrographs at various resolutions and an XRD spectrum of a collagen-inorganic scaffold in accordance with another embodiment of the present invention;

FIG. 9 is a graph showing XRD patterns for two collagen-inorganic is scaffolds in accordance with the invention compared with that of natural bone and carbonated hydroxyapatite;

FIG. 10 is a graph showing XRD patterns for four collagen-inorganic scaffolds in accordance with the invention compared with that of calcium carbonate and brushite;

FIG. 11 is the FTIR spectrum of various starting materials, collagen-inorganic scaffolds in accordance with one embodiment of the present invention and natural trabecular bone.

FIG. 12 is a set of Scanning Electron Micrographs of MC3 T3 (mouse osteoblasts) cells cultured for 1 week on a scaffold in accordance with one embodiment of the present invention;

FIG. 13 is a photograph of a 60 day ectopic implantation of a scaffold in accordance with the present invention into a SCID mouse;

FIG. 14 is a micrograph of implanted scaffold tissue in accordance with the present invention stained with hematoxylin and eosin after 8 days;

FIG. 15 is a micrograph of implanted scaffold tissue in accordance with the present invention stained with hematoxylin and eosin after 30 days;

FIG. 16 is a micrograph of implanted scaffold tissue in accordance with the present invention stained with von Kossa after 8 days;

FIG. 17 is a micrograph of implanted scaffold tissue in accordance with the present invention stained with von Kossa after 30 days;

FIG. 18 is a set of photographs of a Wistar rat femur having a scaffold in accordance with the present invention implanted for six months; and

FIGS. 19A, 19B and 19C are X-rays of Wistar rat femur having a scaffold in accordance with the present invention at 5 months post-implantation (FIG. 19A) and that of Wistar rat femur having a commercial scaffold (FIG. 19B) or no scaffold at all (FIG. 19C).

### DETAILED DESCRIPTION OF PREFERRED EMBODIMENTS

There is provided herein a porous biomaterial-filler composite, the composite comprising biomaterial interspersed with a filler. The present invention provides in one embodiment, a composite comprising a biomaterial phase and inorganic filler interspersed therein. Suitably a polymer phase with an inorganic filler interspersed therein. The biomaterial and the interspersed filler may be chemically bonded to each other.

In one embodiment the composite has a porosity that closely matches that of natural bone. It is desirable in the composite of the present invention in some embodiments to match natural bone as closely as possible, structurally, chemically and mechanically so that a body, in which the composite is implanted can recognize and remodel the composite similarly to natural bone.

In one embodiment the composite has pores having a size greater than about 100 microns, pores having a size between about 30 and 50 microns and small nanometer size pores. The small nanometer-sized pores may be made by voids between filler particles. Such porosity may closely match the actual structure of bone, the large pores enabling osteoblast migration, the medium pores enabling transport of blood/proteins/fluids and the small pores providing traction for better cell attachment.

In one embodiment, the composite contains at least about 25 wt % biomaterial. The filler may be up to 75 wt %. In another embedment the composite contains about 25 to about 50 wt % (dry weight) biomaterial with the amount of filler being between about 35 to about 75 wt % (dry weight). The amounts and biomaterial and filler used may be tailored to have an organic:inorganic ratio to match the composition of natural bone. For example 30 to 35 wt % collagen and 65-70% hydroxyapatite.

In one embodiment, the filler may have previously been prepared by a sol-gel method. Use of a filler prepared in this way may enable pores of nanometer porosity.

There is also provided herein in one embodiment a method of preparing a porous biomaterial-filler composite comprising
combining biomaterial, a liquid and a filler to form a mixture,
homogenizing the mixture to form a slurry, and freeze-drying the slurry to form a porous biomaterial-filler composite.

There is also provided herein in another embodiment a method of preparing a porous biomaterial-filler composite comprising
combining biomaterial and a liquid to form a mixture;
homogenizing the mixture to form a slurry,
adding a filler to the slurry;
further homogenizing the slurry; and
freeze-drying the slurry to form a porous biomaterial-filler composite.

There is also provided herein in another embodiment a method of preparing a porous biomaterial-filler composite comprising
combining filler and a liquid to form a mixture,
homogenizing the mixture to form a slurry,
adding a biomaterial to the slurry;
further homogenizing the slurry; and
freeze-drying the slurry to form a porous biomaterial-filler composite.

In one embodiment, the composite contains at least about 25 wt % biomaterial. In one embodiment the liquid is an acid and the amount of biomaterial in the slurry is in the range of up to about 10 g biomaterial per 100 ml of an up to 1000 mM acid. For example 0.6 to 6 g biomaterial such as collagen per 100 ml of 50 to 500 mM acid such as phosphoric acid (6 mg/ml to 60 mg/ml) results in a composite having different mechanical properties and microstructure.

Examples of some sample compositions as starting materials include:
100 ml of 50 mM phosphoric acid+2 g collagen+7.5 g CAP (carbonated apatite).
100 ml of 50 mM phosphoric acid+2 g collagen+12 g HAP (hydroxyapatite)
100 ml of 100 mM phosphoric acid+2 g collagen+10 g CaCO₃ (calcium carbonate)
100 ml of 50 mM phosphoric acid+4 g collagen+7.5 g CAP (carbonated apatite)
100 ml of 50 mM phosphoric acid+4 g collagen+12 g HAP (hydroxyapatite)
100 ml of 500 mM phosphoric acid+4 g collagen+10 g CaCO₃ (calcium carbonate).

In one embodiment, the biomaterial is a material extracted from biological tissue, including for example, fetal tissue, skin/dermis, muscle or connective tissue, including bone, tendon, ligament or cartilage. In one embodiment the biomaterial is a biopolymer. A biopolymer is suitably a naturally occurring polymeric substance in a biological system or organism. Biopolymers can also suitably be man-made polymers prepared by manipulation of a naturally occurring biopolymer.

Any biomaterial that may be formed into a stable solid structure at body temperature, for example dried into a film, solidified from a melt, cross-linked into a gel, freeze-dried into a foam may be used in the present invention.

In one embodiment the biomaterial is selected from one or more of proteins, peptides, polysaccharides or other organic substances. For example the biomaterial may be selected from one or more of extracellular matrix proteins such as fibronectin, laminin, vitronectin, tenascin, entactin, thrombospondin, elastin, gelatin, collagen, fibrillen, merosin, anchorin, chondronectin, link protein, bone sialoprotein, osteocalcin, osteopontin, epinectin, hyaluronectin, undulin, epiligrin and kalinin, proteoglycans such as decorin, dermatin sulfate proteoglycans, keratin, keratin sulfate proteoglycans, aggrecan, chondroitin sulfate proteoglycans, heparin sulfate proteoglycans, biglycan, syndecan, perlecan, serglycin, glycosaminoglycans such as heparin sulfate, chondroitin sulfate, dermatin sulfate, keratin sulfate or hyaluronic acid, polysaccharides such as heparin, dextran sulfate, chitin, alginic acid, pectin or xylan, polyvinyl alcohol, cytokines, glycosides, glycoproteins, polypyrroles, albumin, fibrinogen, or a phospholipid.

In one embodiment the biomaterial is collagen. In one further embodiment the collagen is one or more selected from the group consisting of collagen Type I, collagen Type II, collagen Type III, collagen Type IV, collagen Type V, collagen type VI, collagen Type VII, collagen Type VIII, collagen Type IX, collagen Type X, collagen Type XI, collagen Type XII, collagen Type XIII, collagen Type XIV, or mixtures thereof. In one embodiment the collagen is Type 1 collagen.

The biomaterial may be extracted from a source by means of acid extraction, salt extraction, enzyme/pepsin extraction or a combination thereof or by some other means such as mechanical extraction for example by grinding. The biomaterial may be prepared by acid extraction followed by precipitating the biomaterial (such as collagen) with sodium chloride and resolubilizing the biomaterial (such as collagen) in a medium having an acidic pH.

Sources of biomaterials include both land and marine vertebrates and invertebrates, for example a mammal, marsupial, a human, a non-human primate, murine, bovine, ovine, equine, caprine, leporine, avian, feline, porcine or canine. In one embodiment the biomaterial is sourced from a mammal or marsupial such as a human, pig, cow, sheep, deer, goat, horse, donkey, hare, rat, mouse, rabbit, kangaroo, wallaby or camel.

In one embodiment, the composite may be formed into a foam, gel or other construct including fibres. In one embodiment the composite is in the form of a porous foam. The foam may include a network of communicating microcompartments with biomaterial molecules and/or filaments interspersed throughout. In an alternative embodiment, the composite may be in the form of biomaterial filaments or fibres having an inorganic filler interspersed therein. In one embodiment the composite is in the form of a cross-linked spongy foam. In another embodiment the composite is in the form of a stiff foam. In one embodiment, the composite may be in the form of a scaffold. A scaffold is a substratum which may be used for anchoring cells.

In one embodiment the filler comprises one or more inorganic compounds. In one embodiment the filler is selected to improve the compressive modulus of the composite. In one embodiment, the inorganic filler comprises calcium carbonate or a calcium-containing salt. In another embodiment, the inorganic filler comprises calcium phosphate, such as an apatite or substituted apatite. In a further embodiment the filler comprises a combination of calcium carbonate and a calcium phosphate such as an apatite. In one embodiment, the calcium phosphate is brushite, tricalcium phosphate, octacalcium phosphate. In various embodiments an apatite is selected from hydroxyapatite (HAP), fluoroapatite (FAP), carbonated apatite (CAP) or zirconium hydroxyapatite (ZrHAP). Apatites containing dopants and additives or substituted apatites may also be used. The filler may suitably be in powder form. In one embodiment the filler is made using a sol-gel method. The sol-gel method is such as described in “Nanostructure Processing of Hydroxyapatite-based Bioceramics”, E S Ahn, N J Gleason, A. Nakahira, J Y Ying Nano Letters 2001 1 (3) p 149-153 which is hereby included by a cross-reference. Producing filler by use of a sol-gel method may enable nanometer-sized grains to be formed and which in the final composite may form small nanometer-sized pores from the voids between the apatite particles. In one embodiment a carbonated apatite prepared by the sol-gel method having a grain size of between about 8 to about 20 nm (for example grains of 8, 9, 10, 11, 12, 13, 14, 15, 16, 17, 18, 19 or 20 nm) or a hydroxyapatite prepared by the sol-gel method having a grain size of between about 40 to about 60 nm (for example 40, 41, 42, 43, 44, 45, 46, 47, 48, 49, 50, 51, 52, 53, 54, 55 56, 57, 58, 59 or 60 nm) may be used. The grain size of the filler may range from 5 nm up to micron range sizes. When the filler is an apatite, the grain size of the filler used may range from 5 to 100 nm.

In another embodiment, the filler is demineralised bone, bone powder, bone morphogenetic protein, calcium sulfate, autologous bone, beads of wax, beads of gelatin, beads of agarose, resorbable polymers or a mixture thereof. These may be used either alone or together and may be used in addition or in place of the apatites and calcium containing compounds.

For preparation of the composite, in one embodiment the liquid is any liquid or fluid capable of forming a slurry. For example the liquid may be water, an organic solvent, an acid, a base or a surfactant. In one embodiment the liquid may be an acid. When the biomaterial is collagen, the liquid may be an acid so as to enable proper dispersion of the collagen. The liquid may include salts or other additives. In a further embodiment the liquid is an inorganic acid. In another embodiment the liquid is an organic acid. For example the liquid may be phosphoric acid. Other acids which may be used include acetic acid, lactic acid, formic acid, tartaric acid, sorbic acid, sulfuric acid, hydrochloric acid, phosphoric acid, ascorbic acid, propanoic acid, triflic acid, trifluoroacetic acids or other acid. Where the composite involves incorporation of calcium carbonate, it is desirable to use phosphoric acid since the reaction between calcium carbonate and an acid other that phosphoric acid may not produce desirable calcium phosphates.

The acid used may have a concentration of up to 1000 mM, for example about 1 mM up to about 500 mM. As further examples 1 mM, 5 mM, 10 mM, 20 mM, 30 mM, 40 mM, 50 mM, 60 mM, 70 mM, 80 mM, 90 mM, 100 mM, 150 mM, 200 mM, 300 mM, 400 mM, 500 mM, 600 mM, 700 mM, 800 mM, 900 mM or 1000 mM acid. In one embodiment the acid has a concentration of up to about 150 mM. In another embodiment the acid has a concentration of up to about 100 mM. Where the filler is calcium carbonate, suitably about 1 mM to 50 mM, for example 100 mM phosphoric acid is used. Where the filler is an apatite, suitably about 1 mM to about 500 mM, for example 50 mM phosphoric acid is used.

In one embodiment the liquid is phosphoric acid and the filler is calcium carbonate. In this embodiment, the phosphoric acid may react with the calcium carbonate to form calcium phosphates which may be precipitated onto the biomaterial.

In one embodiment, when the liquid is an acid, the slurry may include up to about 10 g of the biomaterial per 100 ml of the acid, alternatively up to about 9 g, up to about 8 g, up to about 7 g, up to about 6 g, up to about 5 g, up to about 4 g, up to about 3 g, up to about 2 g, up to about 1 g or up to about 0.5 g biomaterial per 100 ml of the acid. In one embodiment the slurry includes about 0.6 g biopolymer per 100 ml of the acid. In another embodiment the slurry includes about 2 g biopolymer per 100 ml of the liquid. By controlling the ratio of biomaterial to liquid it is possible to modify or control the mechanical properties and microstructure of the composite.

In one embodiment the amount of filler used is selected so that the ratio of biomaterial:powder is similar to that present in the biological system into which the composite is to be inserted. In one embodiment 5 to 15 g of filler is used per 100 ml of liquid. Alternatively about 6 g, about 7 g, about 8 g, about 9 g, about 10 g, about 11 g, about 12 g, about 13 g, about 14 g of filler are used per 100 ml of liquid. In one embodiment a first composite/scaffold may be prepared comprising 100 ml liquid, 0.6 g collagen and filler in the range 5 to 15 g to give a scaffold with a higher porosity due to the higher liquid:solid ratio. In another embodiment a second composite/scaffold may be prepared comprising 100 ml liquid, 2 g collagen and filler in the range 5 to 15 g which may be useful is for insertion as a replacement for bone as it is has lower porosity, is less brittle under compression and has a higher compressive modulus that the first mentioned scaffold.

In one embodiment the mixture is homogenized/dispersed in a mixer. In one embodiment the mixer is a vortex mixer. The mixture may be homogenized/dispersed for from about 1 minute to about 10 hours. In one embodiment the mixture may be homogenized/dispersed for from 10 minutes to about 6 hours. For example, the mixture may be homogenized/dispersed for about 1 hour. As further examples, the mixture may be homogenized/dispersed for 5, 10, 20, 30, 40, 50, 60 minutes, 1, 2, 3, 4, 5, 6, 7, 8, 9 or 10 hours. The slurry may be homogenized/dispersed at 1000 to 60000 rpm. In one embodiment the mixture is homogenized/dispersed at 6000 to 5000 rpm, for example at about 14,000 rpm to form the slurry. Other examples of speeds of the mixer are 1000, 2000, 3000, 4000, 5000, 6000, 7000, 8000, 9000, 10000, 11000, 12000, 13000, 14000, 15000, 16000, 17000, 18000, 19000, 20000, 30000, 40000, 50000, 60000 rpm. The mixture may be cooled, for example, in an ice bath during homogenization, for example at 4° C. Other examples of temperature for cooling include 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C. or 30° C. In one embodiment the mixture is further subjected to sonication to improve homogeneous dispersion.

In accordance with the process of the present invention, the slurry is freeze-dried. By freeze-drying porosity is created in the composite. Porosity may facilitate cell attachment and mobility and may assist blood vessels to infiltrate to allow fluid transport through the scaffold. In one embodiment the pore size and porosity may be controlled by controlling the freezing rate and/or water content of the homogenized slurry prior to freeze-drying. In another embodiment pore size and porosity may be controlled by selection of suitable starting materials and their amounts. In embodiments where an acid such as phosphoric acid and a carbonate such as calcium carbonate are respectively used as liquid or filler, additional porosity may be achieved by production of carbon dioxide bubbles during the reaction between the acid and carbonate.

In one embodiment the composite is subjected to two freezing rates (−80° C. or −20° C. freezer). The freeze-drying protocol may be optimized to control the pore structure, for example, the number and size of pores.

In one embodiment the slurry from the ice bath which may be at 4° C. is poured onto a tray and placed in a freezer held at temperature which may be in the range 0° C. to −50° C., for example −5° C., −10° C., −15° C., −20° C., −25° C., −30° C., −35° C., −40° C., −45° C. In one embodiment the slurry is poured in a tray and placed in an about −20° C. freezer and left to freeze for between 1 to 12 hours depending on the quantity of the mixture. For example the slurry may be frozen for 1, 2, 3, 4, 5, 6, 7, 8, 9, 10, 11 or 12 hours. The frozen mixture may be freeze-dried until a dry porous solid foam is obtained using the following evacuation and heating phases:

Evacuation phase: The freeze-dryer used may be set up so that the condenser temperature is suitably between about 0° C. and about −105° C. For example between about −40 and about −75° C. The vacuum in the freeze-dryer used may be pulled until it is between about 4.58 to about 0.005 torr (about 0.61 kPa-about 0.00067 kPa). For example between about 0.15 to about 0.035 torr (about 0.012-about 0.0047 kPa).

Heating phase: The frozen mixture may be brought from freezing temperature, for example −20° C. into a room temperature environment while simultaneously subjecting to the abovementioned vacuum. This may enable the ice crystals to sublimate, leaving behind pores.

In one embodiment the slurry is held at a temperature of about 0° C. to about 30° C., for example about 5° C., about 10° C., about 15° C., about 20° C., about 25° C. for from 0 to about 60 minutes, for example about 10, about 15, about 20, about 25, about 30, about 35, about 40, about 45, about 50, about 55 minutes. The temperature is then suitably reduced at a ramp rate of from about <1 to about 50° C./min, for example at a ramp rate of about 1°, about 5°, about 10°, about 15°, about 20°, about 25°, about 30°, about 35°, about 40°, about 45° C./min to a final temperature in the range from about −5° C. to about −80° C., for example about −10, −15, −20, −25, −30, −35, −40, −50, −60, −70° C. This final temperature is suitably held for from about 5 minutes up to about 12 hours or more. In one embodiment the freeze-dryer may suitably be set up so that the condenser temperature is between about −105° C. to about 0° C., for example between about −40 C and about −75° C. Vacuum may be pulled until it is between about 4.58 torr (0.61 kPa) and about 0.005 torr (0.00067 kPa), for example between about 0.15 torr (0.12 kPa) and 0.035 torr (0.0047 kPa).

In one embodiment the composite is freeze-dried until substantially all water has been sublimated.

The composite produced may be further processed by the addition of for example coating materials so as to make the properties of the composite similar to that of natural bone. For example the biomaterial may be polymerized or the slurry may be treated with an enzyme such as lysyl oxidase. In another embodiment the biomaterial may be esterified, acylated, deaminated or blocked with a blocking agent. Additives may be added to the slurry. For example fibre reinforcement, polypeptides, glycoprotein antifreezes, pharmaceuticals, antibiotics, growth factors or bone morphogenetic protein may be added to the slurry.

In one embodiment the composite may be conditioned with cells. Suitable cells include, but are not limited to, epithelial cells such as keratinocytes, adipocytes, hepatocytes, neurons, glial cells, astrocytes, podocytes, mammary epithelial cells, islet cells, endothelial cells such as aortic, capillary and vein endothelial cells, and mesenchymal cells such as dermal fibroblasts, mesothelial cells, stem cells, osteoblasts, smooth muscle cells, striated muscle cells, ligament fibroblasts, tendon fibroblasts, chondrocytes or fibroblasts.

In one embodiment, the freeze-dried composite is cross-linked. Cross-linking may increase the compressive modulus of the composite and improve its resistance to degradation. Cross-linking may cause the composite to become more physically stable and insoluble in aqueous medium. In one embodiment the composite/scaffold degradation rate can be controlled by varying the extent of cross-linking in the composite such as described by Y. S. Pek et al. in Biomaterials 25 (3) (2004) p 473-482. For example the freeze-dried composite may be cross-linked so that it forms a stiff foam or a spongy foam or an intermediate between a stiff foam and spongy foam.

In one embodiment the freeze-dried product may be subjected to cross-linking. In another embodiment, the slurry may be cross-linked prior to freeze-drying. Cross-linking may be performed by a cross-linking method such as by amide cross-linking. Example 1 describes one method of amide cross-linking using EDC/NHS.

Other cross-linking methods or cross-linking agents may be used. For example, physical, chemical and enzymatic methods of cross-linking may be used. Cross-linking may be performed with acrylamides, diones, glutaraldehyde, acetaldehyde, formaldehyde or ribose. UV or other irradiation methods such as gamma irradiation, dehydrothermal methods may be used.

Compared with current scaffolds on the market, the scaffolds of the present invention have better mechanical properties and microstructural and chemical match to natural bone, and are more osteoinductive.

The composite/scaffold of the present invention has the advantage that it has sufficient mechanical strength for load bearing applications and sufficient bioactivity to promote attachment and proliferation. The material of the invention may be resorbable by natural tissue and tunable to mechanical properties and degradation rates. The material of the invention may have microporosity of the order of about 100 to 600 microns, for example=200 microns. The material is biocompatible and capable of being is resorbed and replaced by tissue. The composite/scaffold in one embodiment of the invention has porosity equivalent to that of bone.

The composite of the present invention can be used in methods of replacing or repairing bone by implanting the composite.

The composite/scaffolds are suitable for orthopedic or other load-bearing applications. The scaffold can be used as an osteoinductive load-bearing hard tissue implant and can be also used for other tissue engineering applications. The scaffolds can be used as a resorbable bone substantive to aid in healing of large fractures and bone loss. The scaffolds have sufficient strength to support the daily activities of the host animal during recovery. They demonstrate sufficient bioavailability in vivo for rapid cell attachment. The scaffold can be used in tissue repair or reconstruction enabling regeneration of replacement tissue, for dressings, as hemostatic agents or as a support for cell growth in vivo and in vitro. The scaffold can also be used as a carrier containing protein or drugs for delivery. The scaffold can be used as a model system for research or as prostheses or implants to replace damaged or diseased tissues or to provide scaffolds which when occupied by cells are remodeled to become functional tissues. The scaffold can be seeded with cells and can be seeded with cells of the same type as those of the tissue which the scaffold is used to repair, reconstruct or replace. The scaffold may also be seeded with stem cells. The scaffold can be used as a prosthesis or implant and can be used to replace tissue such as skin, nervous tissue, vascular tissue, cardiac tissue, pericardial tissue, muscle tissue, ocular tissue, periodontal tissue, connective tissue such as a bone, cartilage, tendon or ligament, organ tissue, liver tissue, glandular tissue, mammary tissue, adrenal tissue, urological tissue and digestive tissue. The scaffold can be used as an implant which can be introduced or grafted into a suitable recipient such as a mammal including a human. The scaffold can also be used as a dressing such as a skin dressing or for drug delivery.

The composite/scaffold of the present invention may be applied topically, subcutaneously, intraperitoneally or intramuscularly.

The invention will now be described in greater detail by reference to the following specific examples, which should not be construed in any way as limiting the scope of the invention.

### EXAMPLE A

The following example is a description of a method of preparing a hydroxyapatite which may be used in the present invention. The hydroxyapatite in this method is prepared by the sol-gel process.

The following starting materials were used:

| | |
|---|---|
| Ca(NO₃)₂•4H₂O | mw = 236.15 |
| (NH₄)₂HPO₄ | mw = 132.06 |

A first solution containing 0.05 M to 0.5 M calcium nitrate Ca(NO₃)₂.4 H₂O in dH₂O (deuterated water) was prepared. Separately a second solution containing 0.05 M to 0.5 M ammonium phosphate (NH₄)₂ HPO₄ in dH₂O was prepared to which was added a suitable amount of an ammonia solution NH₄OH to adjust the pH of solution to about 10. The first solution was then added dropwise to the second solution. The solution was then aged for 100 hours at room temperature and a precipitate was then collected by centrifuging: The precipitate was then washed with three portions of decreasing concentrations of an ammonia solution NH₄OH and dH₂O, followed by two ethanol washes.

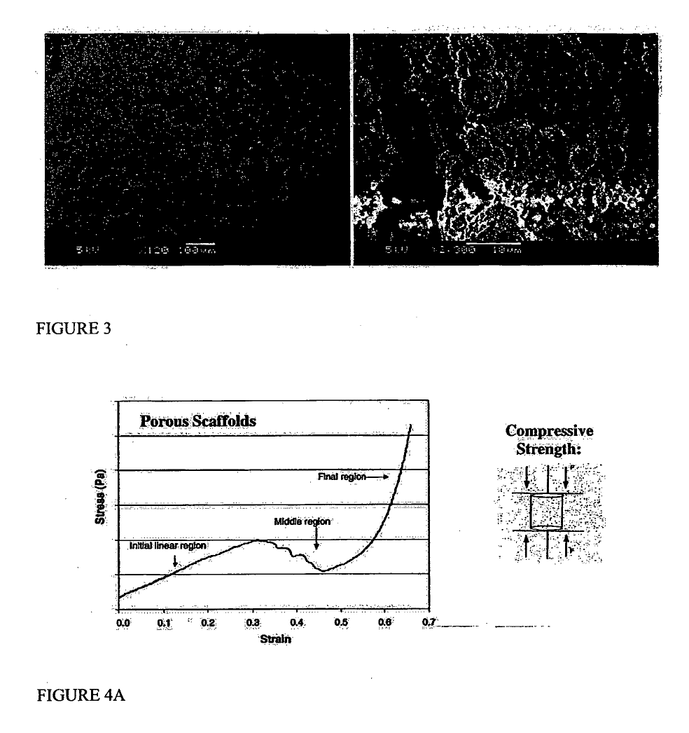

The resulting gel was air-dried for 24 hrs on a watchglass, and further oven dried for 12 hrs at 120° C. The powder was ground and the resulting ground powder was then dried on a hot plate and optionally calcined at above 500° C. for 5 hours. To determine the microstructure, a scanning electron micrograph of a dry hydroxyapatite produced by this method was undertaken and the results are shown in FIG. 3 at resolutions of ×120 and ×2.3. The grains were approximately 25 nm (by XRD X-ray diffraction) aggregated to form agglomerates.

### EXAMPLE B

The following example is a description of a method of preparing a carbonated apatite which may be used in the present invention. The carbonated apatite in this method is prepared by the sol-gel process as follows.

The following starting materials were used:

| | |
|---|---|
| Ca(NO₃)₂•4H₂O | mw = 236.15 |
| (NH₄)₂HPO₄ | mw = 132.06 |
| (NH₄)HCO₃ | mw = 79.06 |

A first solution containing 0.05 M to 0.5 M calcium nitrate Ca(NO₃)₂.4 H₂O in dH₂O (deuterated water) was prepared. Separately a second solution containing 0.05 M to 0.5 M ammonium phosphate (NH₄)₂ HPO₄, 0.05 M to 0.5 M ammonium carbonate (NH₄)HCO₃ and a suitable surfactant in dH₂O was prepared. A suitable amount of an ammonia solution NH₄OH was added to adjust the pH of the solution to about 10. The first solution was then added dropwise to the second solution. The solution was then aged for 100 hours at room temperature. A precipitate was then collected by centrifuging. The precipitate was then washed with three portions of decreasing concentrations of ammonia solution NH₄OH and dH₂O, followed by two ethanol washes.

The resulting gel was air-dried for 24 hrs on a watchglass, and further oven dried for 12 hrs at 120° C. The powder was ground and the resulting ground powder was then dried on a hot plate.

### EXAMPLE 1

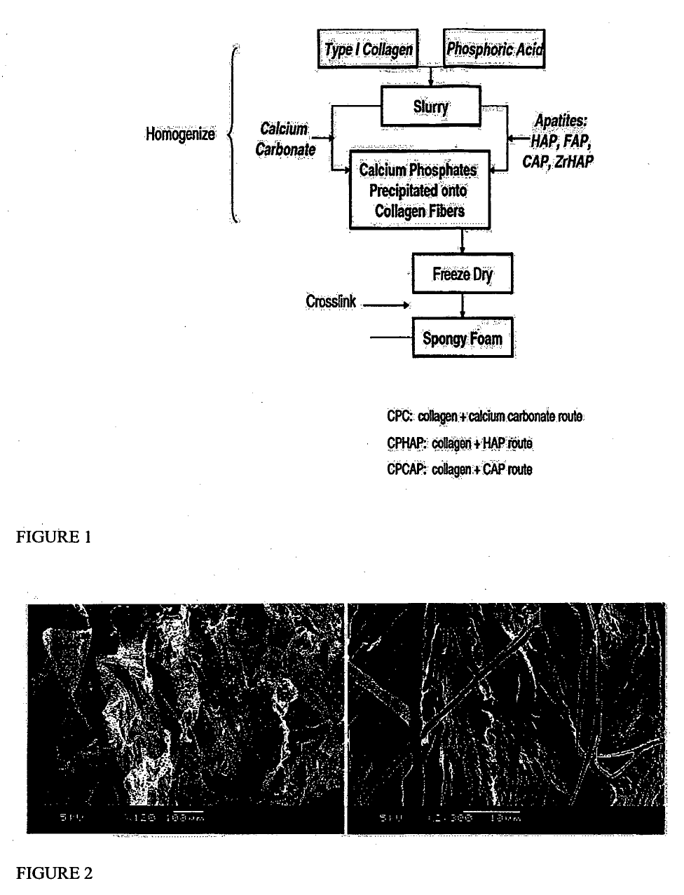

FIG. 1 shows a schematic diagram for producing a scaffold in accordance with one embodiment of the present invention. In this embodiment Type 1 collagen is used such as shown in FIG. 2. FIG. 2 is a set of scanning electron micrographs of a dry collagen suitable for use in the present invention at resolutions of ×120 and ×2.3. The microstructure shows fibrils of approximately 1.5-2 μm in diameter mixed with thin filmy sheets.

As shown in FIG. 1, Type 1 collagen and phosphoric acid are combined into a slurry and homogenized and either calcium carbonate and/or an apatite (such as HAP, FAP, CAP, or ZrCAP) added to the slurry. The slurry is then homogenized whereby calcium phosphate and/or the apatite are interspersed and precipitated onto the collagen fibers.

The slurry is then freeze dried followed by cross-linking to form a spongy foam. The freeze-dried product may be crosslinked according to the following protocol:

1-ethyl-3-(3-dimethylaminopropyl)carbodiimide/N-hydroxysuccinimide (EDC/NHS) crosslinking protocol—modified from the protocol of Olde Damink et al., Biomaterials 17 (1996) 765-773.

Because EDC has a molecular weight of 197 g/mol, 0.276 g is used per 100 ml, for s example. Because NHS has a molecular weight of 115 g/mol, 0.064 g is used per 100 ml, for example.
(1) The freeze-dried matrices of the invention are hydrated in half the final volume of deionized water (for example hydrated in 50 ml of sterile deionized water for 100 ml final volume of freeze-dried product).
(2) This is followed by dissolving EDC and NHS in half the final volume of deionized water (for example dissolve in 50 ml of sterile deionized water for 100 ml of final volume). This solution is suitably made up fresh for each use. The final concentration is 0.014 M EDC and 0.005 M NHS;
(3) Sterile filter the EDC/NHS solution through a 0.2 mm filter into a sterile container (or directly into a container containing the hydrated matrices). Suitably use 6 mmol EDC/g collagen with an EDC:NHS ratio of 5:2;
(4) Cross-link at room temperature for about 2 hours;
(5) Discard solution as hazardous waste;
(6) Rinse matrices in sterile PBS (phosphate buffer saline solution), change to fresh, sterile PBS and incubate for about 2 hours;
(7) Rinse for about 2×10 minutes-twice in sterile deionized water; and
(8) Store at 4° C. for up to one week before use.

Cross-linked scaffolds were produced as described.

The effect of the following synthesis parameters on the final product were compared:
(i) Effect of freezing rate on microstructure (e.g. pore characteristics) and mechanical properties (eg compressive modulus);
(ii) Effect on inorganic powder:slurry ratio on microstructure (e.g. pore characteristics) and mechanical properties (eg compressive modulus) and chemical composition and crystalline phase;
(iii) Effect of EDC/NHS cross-linking on microstructure (e.g. pore characteristics) and mechanical properties (eg compressive modulus).

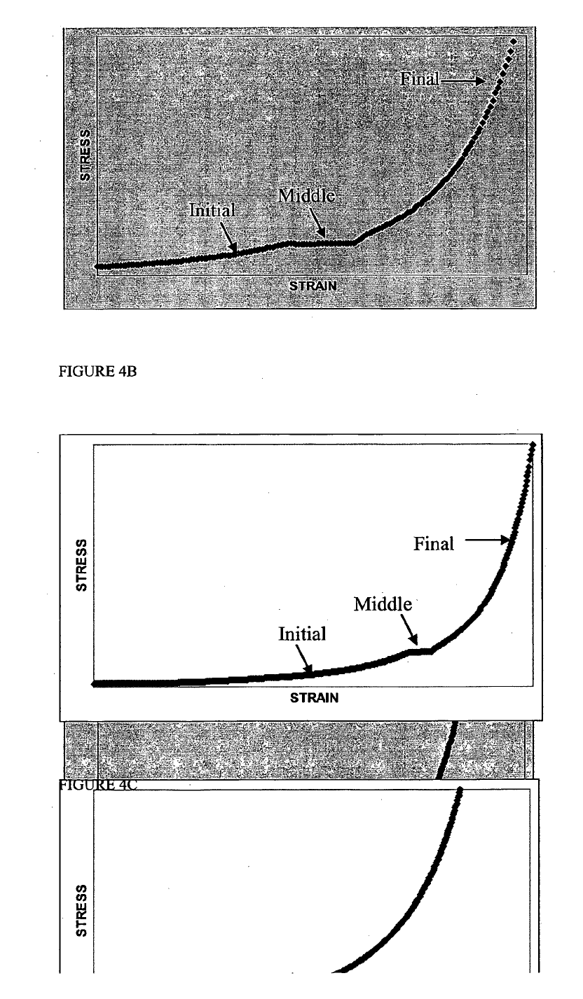

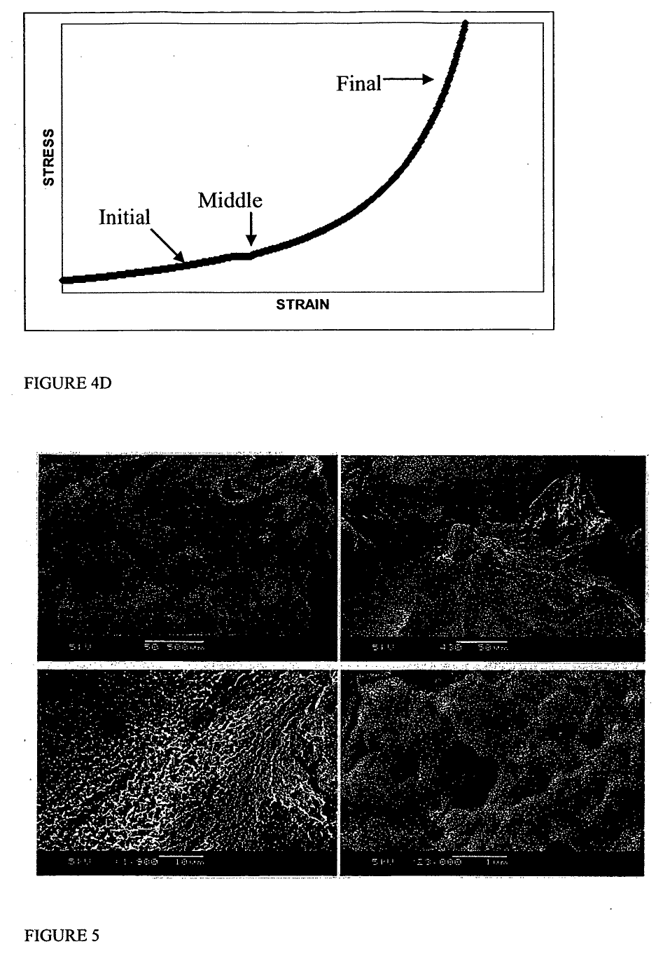

A number of collagen-inorganic scaffolds were prepared using various freezing rates, powder:slurry ratios and with optional cross-linking. Compression tests were done in Simulated Body Fluid (SBF) at 37° C. according to British Standard 6039:1981 for orthopedic and dental materials. Each compression point was obtained from an average of eight samples. Initial compressive modulus was obtained from compression of pores, final compressive modulus was obtained from compression of the bulk material after the pore had collapsed. Results of the compression tests are shown in FIGS. 4A to 4B. FIG. 4A is a compression curve for a scaffold produced using 100 ml of 50 mM phosphoric acid, 4 g collagen and 10 g hydroxyapatite. FIG. 4B is a compression curve for a scaffold produced using 100 ml of 50 mM phosphoric acid, 2 g collagen and 5 g carbonated apatite. FIG. 4C is a compression curve for a scaffold produced using 100 ml of 50 mM phosphoric acid, 2 g collagen and 5 g hydroxyapatite. FIG. 4D is a compression curve for a scaffold produced using 100 ml of 100 mM phosphoric acid, 2 g collagen and 5 g calcium carbonate. The scaffolds were frozen at −20° C.

Compression tests indicate that a slow freezing rate to a colder final freezing temperature results in larger ice crystals leading to larger pores after freeze-drying. The rate of freezing however has no apparent affect on porosity. Compression tests indicate that the final freezing temperature of −80° C. leads to much lower compressive modulus for all samples compared to −20° C. It is therefore recommended that the freeze-drying temperature does not go below −50° C. Others have reported damage to the collagen structure at −80 to −50° C.—Fois et al., J. Polym. Sci. Part B—Polym. Physics, 38 (7) (2000) 987-992 and this may explain the lower final compressive modulus.

It has been found that the final compressive modulus (100-300 MPa) can be adjusted by varying the powder:slurry ratio. A high powder:slurry ratio results in a high initial compressive modulus: however if this ratio is too high, the collagen matrix cannot hold all the powder and excess powder would leach out during compression leading to no significant improvement or even a decease in the final compression modulus. Suitably weight ratios of power to slurry are up 5-15 g powder:up to 10 g collagen per 100 ml of liquid solution.

It has been found the cross-linking also results in a higher compressive modulus.

### EXAMPLE 2

Various micrographs of collagen-inorganic scaffolds made in accordance with the present invention were obtained and compared to that of trabecular bone.

As shown in FIG. 5 micrographs of trabecular bone at various resolutions is seen to be dense, with some large pores of ˜300-400 μm as well as smaller pores of ˜30-50 μm as well as nanometer-sized pores.

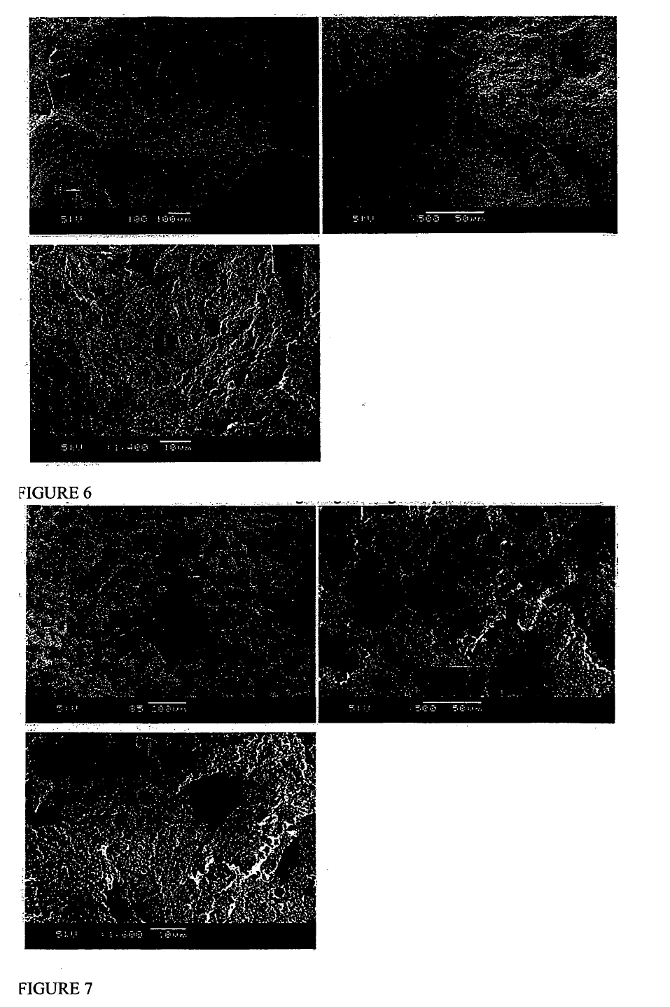

FIG. 6 shows micrographs at various resolutions for a CPCAP scaffold made from 100 ml of 50 mM phosphoric acid, 4 g collagen and 7.5 g CAP (carbonated apatite powder). It can be seen from FIG. 6 that this material has the closest match with bone in microstructure, with large pores of ˜200-300 μm, as well as smaller pores of ˜20-30 μm and nanometer-sized pores from voids between CAP particles.

FIG. 7 shows micrographs at various resolutions for a CPHAP scaffold made from 100 ml of 50 mM liquid), 4 g collagen and 12 g HAP (hydroxyapatite) powder using the same scaffold manufacturing method as described in Example 1. This material is bone-like but requires additional large pores of ˜300 μm (which can be obtained by optimizing the freezing rate), many pores of ˜20-30 μm as well as nanometer-sized pores from voids between HAP particles.

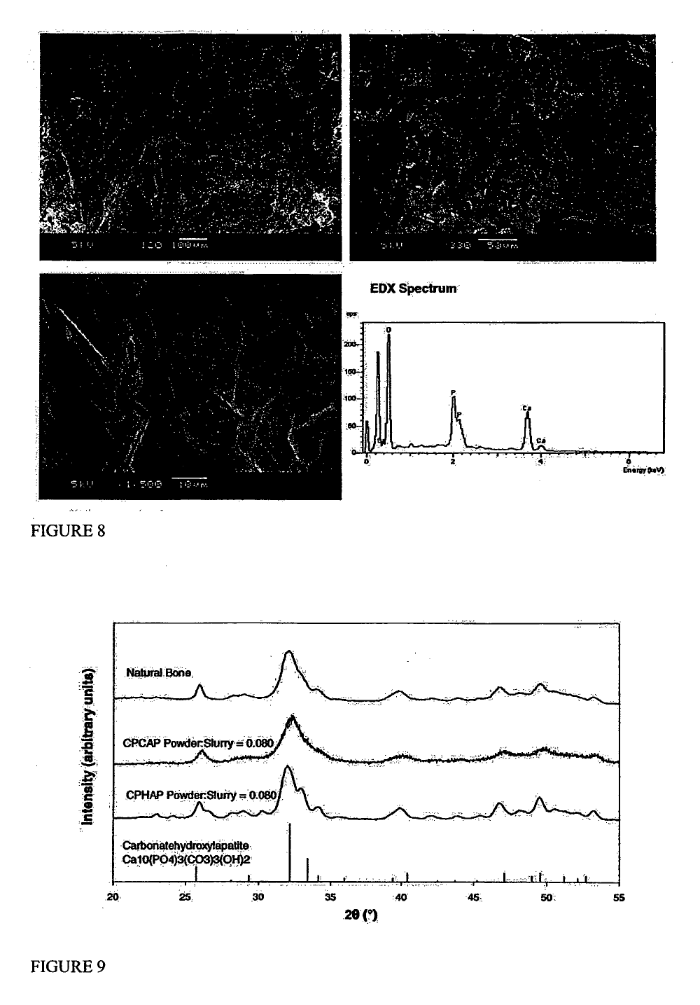

FIG. 8 shows micrographs at various resolutions and an EDX (Energy Dispersive X-ray) spectrum for a CPC scaffold made from 100 ml of 100 mM phosphoric acid, 4 g collagen and 10 g calcium carbonate powder using the method described in Example 1. It can be seen that this material is bone-like but requires more large pores of ˜300 μm (which can be obtained by optimizing the freezing rate). The EDX spectrum shows that the particles are some form of calcium phosphate resulting from the acid-carbonate reaction.

### EXAMPLE 3

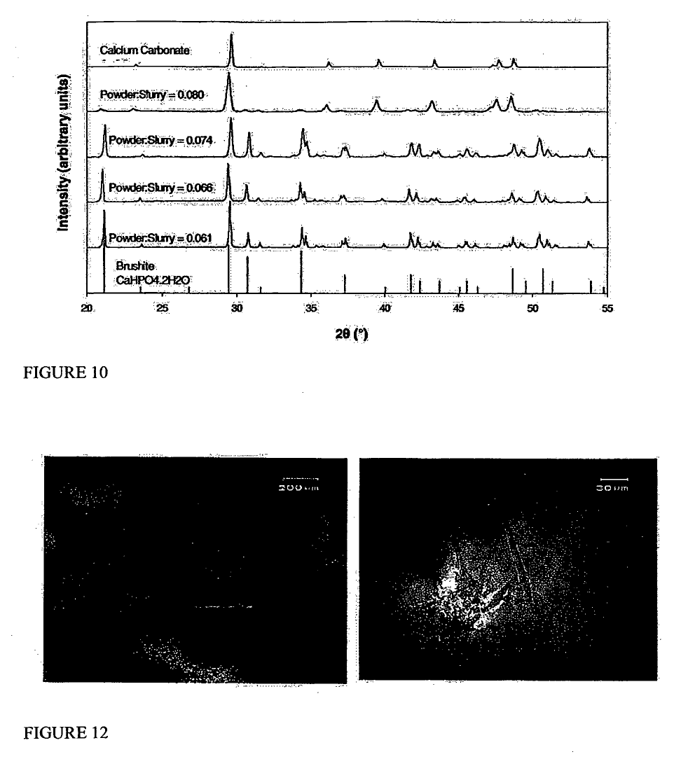

XRD and FTIR studies were undertaken of the scaffold products. FIG. 9 shows XRD patterns for various collagen-carbonated apatite (CPCAP) and collagen-hydroxyapatite (CPHAP) scaffolds made using the method as described in Example 1 and that of natural bone. It is envisaged that the best match with natural bone may be obtained by combining CAP and HAP powders in optimal proportions in the same scaffold. FIG. 10 shows various XRD patterns for collagen-calcium carbonate scaffolds wherein the slurry concentration was 0.6 g collagen per 100 ml of 100 mM phosphoric acid using the general method as described in Example 1. Also shown are the XRD patterns for brushite and calcium carbonate. It can be seen that the XRD pattern varies depending on powder:slurry ratio.

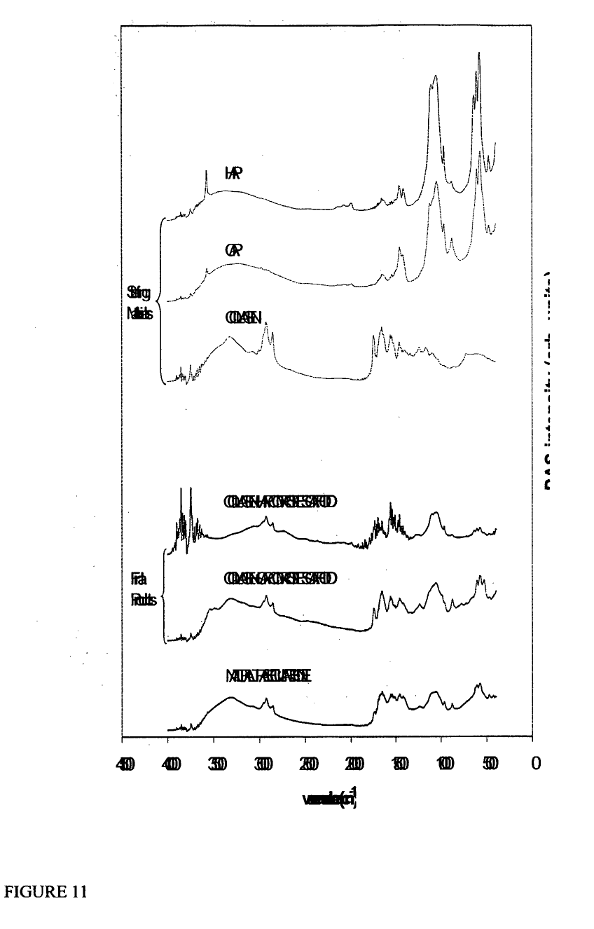

FIG. 11 shows Fourier Transform Infrared spectra (FTIR) of starting materials and scaffolds made by the method of Example 1 and that of natural bone. It can be seen that the scaffolds of the invention closely match in chemical structure to natural bone.

### EXAMPLE 4

In vitro studies with MC3T3 osteoblast cells indicate excellent cell attachment and proliferation on the material of the invention. In this example MC3 T3 (mouse osteoblast) cells was cultured for one week on a scaffold of the invention using a scaffold made from 100 ml of 50 mM phosphoric acid, 0.6 g collagen and 1 g hydroxyapatite. The results are shown in FIG. 12. It can be seen that cells attach to and occupy pore sites within the scaffold.

### EXAMPLE 5

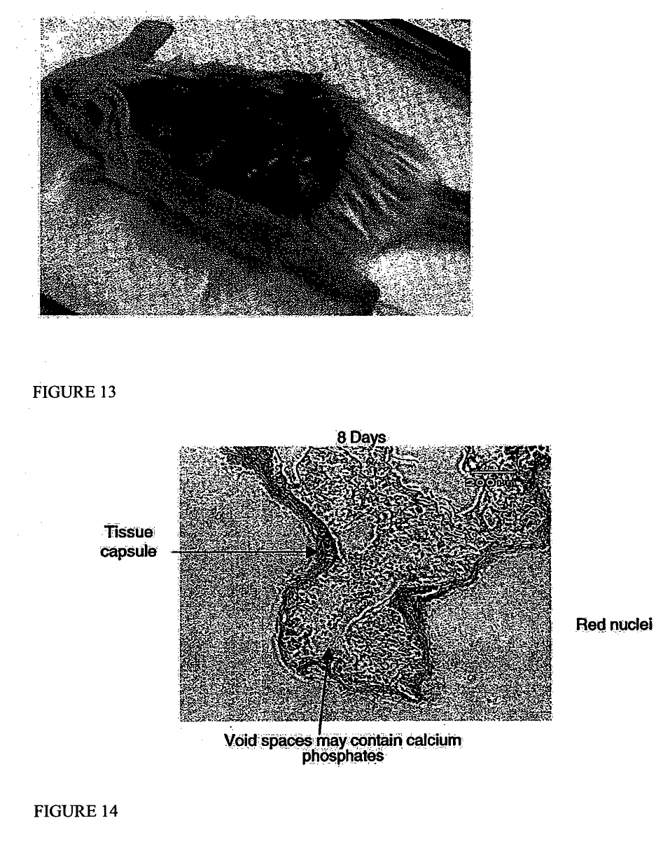

An in vivo study of 60 day ectopic implantation of scaffolds into SCID mice was conducted using a scaffold made from 100 ml of 50 mM phosphoric acid, 2 g collagen and 5 g hydroxyapatite. All animals survived for the full two months of experimentation and no adverse side effects were observed. The results are shown in FIG. 13. This study shows the ectopic in vivo implantation on SCID mice shows evidence of bone, tissue and blood vessel formation in the scaffold material of the invention. The results show that the scaffolds are biocompatible, with sufficient support for tissue in growth, vascularization, osteon formation and bone mineralization. This is confirmed by the histological results shown in FIGS. 14 to 17.

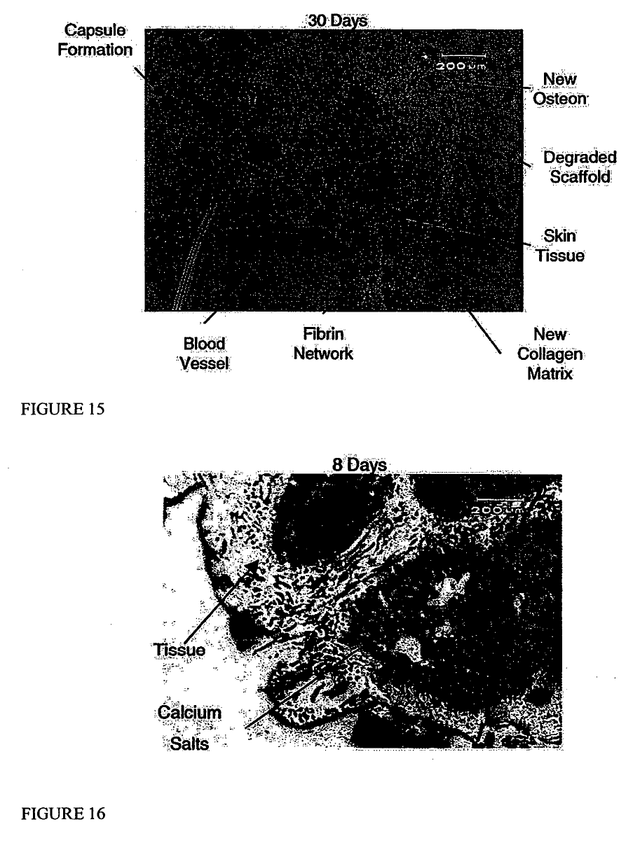

FIGS. 14 and 15 show Hematoxylin and Eosin staining after 8 days and 30 days. It can be seen from the Figures that tissue capsules and void spaces can be recognized which may contain calcium phosphate. In addition capsule formation, new osteon, degraded scaffold, blood vessel formation, fibrin networks, new collagen matrix and skin tissue can be seen.

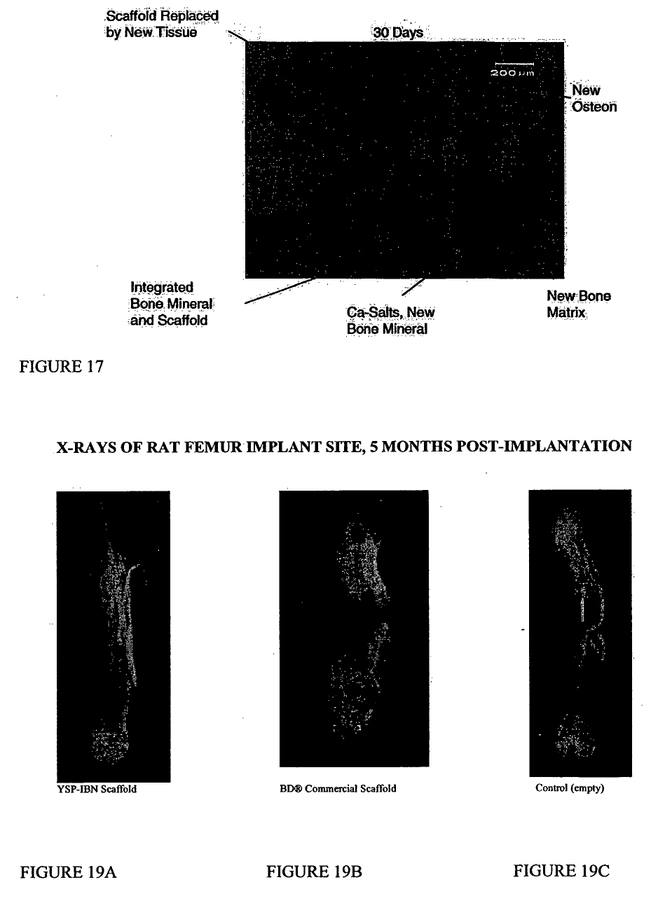

FIGS. 16 and 17 show Von Kossa Staining after 8 and 30 days. It can be seen from the Figures that calcium salts and tissue can be seen. The scaffold is replaced by new tissue, new osteon, new bone matrix, new bone material and integrated bone mineral and scaffold. The study shows in vivo implantation of the scaffold material of the invention in critical-sized defects on Wistar/SD rat femur resulted in successful healing and functioning of the defect area without the need for an external supporting cast. There was successful integration of the scaffolds with the surrounding host tissue.

### EXAMPLE 6

In vivo load-bearing potential of porous collagen-based bone implants of the invention were determined. Porous bone implants were synthesized from collagen and calcium phosphate for bone replacement purposes.

Wistar/SD rates were chosen as this species is one of the most commonly used for in vivo studies of a similar nature. 50 female animals were used having an average weight of 180 to 220 g. The rats used were observed for I week prior to surgery. Just before surgery the rats were anesthetized with Nembutal injection solution by IP (intraperitoneal) using ≦0.1 ml/100 g of animal weight. It was ensured that the animals were properly anesthetized by using the toe pinch test for pain reflexes. Surgery was only performed on one femur for each animal. The operating region was shaved to remove excess surface hair followed by ectopic sterilization with 70% ethanol.

The skin of the animal was cut and muscles denuded to expose the femur. The Protocol for exposing the midshaft femur was as follows:
1. Curved incision just cranial to the femoral shaft;
2. Biceps femoris (BF) encountered if incision was directly over the shaft;
3. Do not incise BF;
4. Incise fascia lata cranial to BF;
5. Retract vastus lateralis cranially;
6. Retract BF caudally; and
7. Transect femur;

A 3 mm length of bone was removed from the mid-section of the femur using a surgical saw (or similar tool as appropriate) to cut completely through the femur.

Scaffolds of appropriate size were inserted in a compressed fit into the gap created by the bone removal. Scaffolds were fixed in position using stainless steel (Kirschner) pins and/or wires.

The procedure was as follows:
1. Pin inserted in retrograde fashion, exiting proximally through the trocanteric fossa;
2. Animals hip extended and the femur adducted on pushing pin through the trochanteric notch to avoid sciatic nerve and insert scaffold; and
3. Figure of Eight wire fixation used for additional support.

Muscle and skin at the trauma site was sutured shut using silk sutures (No. 4).

After surgery, the animals were held in a common room at 22° C. to recover and allowed to consume food and drink as necessary.

The animals were allowed to acclimatise (i.e. pre-operative management) for at least one week. Where necessary Buprenorphine was administered subcutaneously for post-operative pain. The animals were examined daily after surgery for about 3 weeks.

The animals were euthanized using an overdose of carbon dioxide by inhalation.

Preliminary X-rays obtained from scaffolds that were implanted for 2 months showed some bone healing with the fixation pins being removable.

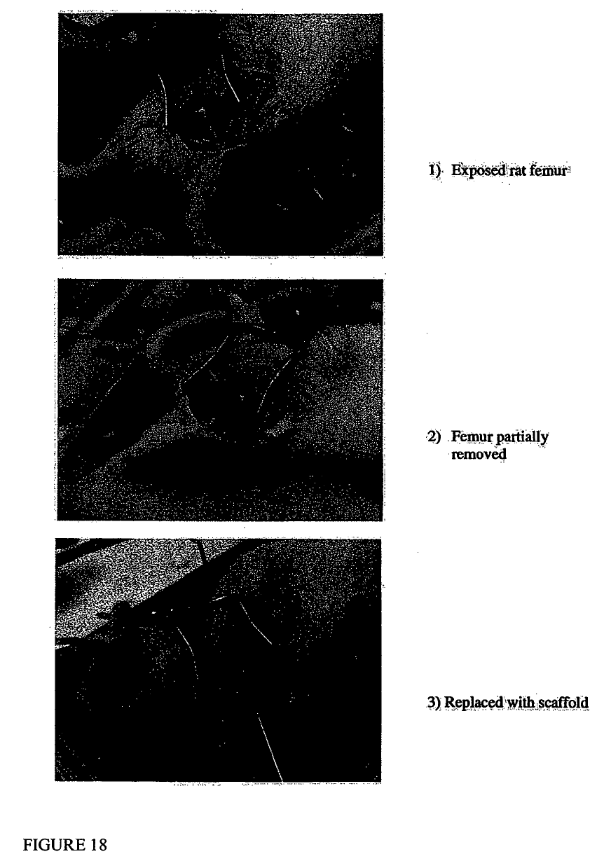

FIG. 18 shows a six month implantation of a scaffold in accordance with the invention implanted by the above method into Wistar rat femur (load bearing). The results show that the scaffolds are suitable resorbable bone substitute materials to aid in healing of large fractures and bone loss. The scaffolds of the invention have sufficient strength to support the daily activities of the host animal during recovery. In addition, they demonstrate sufficient bioavailability in vivo for rapid attachment and proliferation.

A portion of the rats were sacrificed 5 months post-implantation and the femurs removed for X-ray. As shown in FIG. 19, X-rays show that a scaffold in accordance with the present invention (collagen-hydroxyapatite marked YSP-IBN in the Figure) FIG. 19A was a more successful scaffold than that of a commercial scaffold (BD Biosciences) FIG. 19B compared with an empty control FIG. 19C.

### INDUSTRIAL APPLICABILITY

The composite of the present invention can be used in many dental, orthopedic, pharmaceutical, medical, veterinarial applications and can be used amongst others as a haemostatic agent, a scaffold for tissue repair or as a support for cell growth.

It will be appreciated by persons skilled in the art that numerous modifications and/or variations may be made to the invention as shown in the specific embodiments disclosed without departing from the spirit and scope of the invention as broadly described. The specific embodiments disclosed herein are therefore to be considered in all respects as illustrative and not restrictive.

1. A porous biomaterial-filler composite wherein the composite comprises biomaterial interspersed with a filler and wherein the composite has a porosity close to that of natural bone.

2. The composite of claim 1, wherein the composite has pores having a size greater than about 100 microns, pores having a size between about 30 and 50 microns and small nanometer size pores.

3. The composite of claim 1, wherein the composite contains at least about 25 wt % biomaterial.

4. The composite of claim 1, wherein the composite contains about 25 to about 50 wt % (dry weight) biomaterial with the amount of filler being between about 35 to about 75 wt % (dry weight).

5. The composite of claim 1, wherein the composite contains 30 to 35 wt % collagen and 65-70% hydroxyapatite.

6. The composite of claim 1, wherein the filler has previously been prepared by a sol-gel method.

7. The composite of claim 1, wherein the biomaterial is a material extracted from biological tissue selected from one or more of fetal tissue, skin/dermis, muscle or connective tissue.

8. The composite of claim 1, wherein the biomaterial is a biopolymer.

9. The composite of claim 1, wherein the biomaterial is selected from one or more of proteins, peptides, polysaccharides or other organic substance.

10. The composite of claim 1, wherein the biomaterial is selected from one or more of extracellular matrix proteins, fibronectin, laminin, vitronectin, tenascin, entactin, thrombospondin, elastin, gelatin, collagen, fibrillen, merosin, anchorin, chondronectin, link protein, bone sialoprotein, osteocalcin, osteopontin, epinectin, hyaluronectin, undulin, epiligrin, kalinin, proteoglycans, decorin, dermatin sulfate proteoglycans, keratin, keratin sulfate proteoglycans, aggrecan, chondroitin sulfate proteoglycans, heparin sulfate proteoglycans, biglycan, syndecan, perlecan, serglycin, glycosaminoglycans, heparin sulfate, chondroitin sulfate, dermatin sulfate, keratin sulfate, hyaluronic acid, polysaccharides, heparin, dextran sulfate, chitin, alginic acid, pectin, xylan, polyvinyl alcohol, cytokines, glycosides, glycoproteins, polypyrroles, albumin, fibrinogen, or a phospholipid.

11. The composite of claim 1, wherein the biomaterial is collagen.

12. The composite of claim 11, wherein the collagen is one or more selected from the group consisting of collagen Type I, collagen Type II, collagen Type III, collagen Type IV, collagen Type V, collagen type VI, collagen Type VII, collagen Type VIII, collagen Type IX, collagen Type X, collagen Type XI, collagen Type XII, collagen Type XIII, collagen Type XIV, or mixtures thereof.

13. The composite of claim 12, wherein the collagen is Type 1 collagen.

14. The composite of claim 1 in the form of a foam, gel, cross-linked spongy foam, stiff foam or other construct including fibres.

15. The composite of claim 1, in the form of a scaffold.

16. The composite of claim 1 wherein the filler comprises one or more inorganic compounds.

17. The composite of claim 16 wherein the filler comprises:
(i) calcium carbonate or a calcium-containing salt;
(ii) a calcium phosphate; or
(iii) a combination of calcium carbonate and a calcium phosphate.

18. The composite of claim 17 wherein the calcium phosphate is brushite, tricalcium phosphate or octacalcium phosphate.

19. The composite of claim 17 wherein the apatite is selected from hydroxyapatite (HAP), flu oroapatite (FAP), carbonated apatite (CAP) or zirconium hydroxyapatite (ZrHAP).

20. The composite of claim 1 wherein the filler is a carbonated apatite prepared by the sol-gel method having a grain size of between about 8 to about 20 nm or a hydroxyapatite prepared by the sol-gel method having a grain size of between about 40 to about 60 nm.

21. The composite of claim 1 wherein the filler is demineralised bone, bone powder, bone morphogenetic protein, calcium sulfate, autologous bone, beads of wax, beads of gelatin, beads of agarose, resorbable polymers or a mixture thereof.

22. A porous biomaterial-filler composite wherein the composite comprises biomaterial interspersed with a filler and wherein the composite includes pores having a size greater than about 100 microns, pores having a size between about 30 and about 50 microns and nanometer sized pores.

23. A porous biomaterial-filler composite wherein the composite comprises biomaterial interspersed with a filler and wherein the dry weight ratio of biomaterial to filler is in the range 1:3 to 2:1.

24. The composite of claim 23 wherein the composite comprises by dry weight 25 to 65 wt % biomaterial and 35 to 75 wt % filler.

25. A porous biomaterial-filler composite wherein the composite comprises biomaterial interspersed with a filler wherein the amount of biomaterial is at least about 25 wt %.

26. A porous biomaterial-filler composite wherein the composite comprises biomaterial interspersed with a filler and wherein the composite filler has been prepared using a sol-gel method.

27. A method of preparing a porous biomaterial-filler composite comprising
combining biomaterial, a liquid and a filler to form a mixture,
homogenizing the mixture to form a slurry, and
freeze-drying the slurry to form a porous biomaterial-filler composite.

28. A method of preparing a porous biomaterial-filler composite comprising
combining biomaterial and a liquid to form a mixture,
homogenizing the mixture to form a slurry,
adding a filler to the slurry;
further homogenizing the slurry; and
freeze-drying the slurry to form a porous biomaterial-filler composite.

29. A method of preparing a porous biomaterial-filler composite comprising
combining a filler and a liquid to form a mixture,
homogenizing the mixture to form a slurry,
adding a biomaterial to the slurry;
further homogenizing the slurry; and
freeze-drying the slurry to form a porous biomaterial-filler composite

30. A method of preparing a porous biomaterial-filler composite comprising
combining biomaterial, a liquid and a filler to form a mixture,
homogenizing the mixture to form a slurry, and
freeze-drying the slurry to form a porous biomaterial-filler composite, wherein the liquid is an acid and the amount of biomaterial in the slurry is in the range of up to about 10 g biomaterial per 100 ml of up to a 1000 mM acid.

31. A method of preparing a porous biomaterial-filler composite comprising
combining biomaterial and a liquid to form a mixture,
homogenizing the mixture to form a slurry,
adding a filler to the slurry;
further homogenizing the slurry; and
freeze-drying the slurry to form a porous biomaterial-filler composite, wherein the liquid is an acid and the amount of biomaterial in the slurry is in the range of up to about 10 g biomaterial per 100 ml of up to a 1000 mM acid.

32. A method of preparing a porous biomaterial-filler composite comprising
combining filler and a liquid to form a mixture,
homogenizing the mixture to form a slurry,
adding a biomaterial to the slurry;
further homogenizing the slurry; and
freeze-drying the slurry to form a porous biomaterial-filler composite, wherein the liquid is an acid and the amount of biomaterial in the slurry is in the range of up to about 10 g biomaterial per 100 ml of up to a 1000 mM acid.

33. A composite prepared by the method of claim 27, 28,29, 30, 31 or 32.
